(19) United States
(12) Patent Application Publication
(10) Pub. No.: US 2025/0143912 A1
(43) Pub. Date: May 8, 2025

(54) OSTOMY POUCH WITH VIEWING OPTION WINDOW

(71) Applicant: HOLLISTER INCORPORATED, Libertyville, IL (US)

(72) Inventor: Robert J. WEINBERG, Lake Villa, IL (US)

(73) Assignee: HOLLISTER INCORPORATED, Libertyville, IL (US)

(21) Appl. No.: 18/834,093

(22) PCT Filed: Feb. 1, 2023
(86) PCT No.: PCT/US2023/061761

§ 371 Date: Jul. 29, 2024

Related U.S. Application Data
(60) Provisional application No. 63/320,804, filed on Mar. 17, 2022.

(51) Int. Cl. A61F 5/445; A61F 5/44

(52) U.S. Cl. CPC A61F 5/445; A61F 5/4404

(57) ABSTRACT

An ostomy pouch includes a viewing window member formed from an elastomeric material covering at least a portion of a distal pouch wall. The viewing window member includes a viewing opening. The viewing window member is configured to be stretched to increase the size of the viewing opening for viewing an interior of the ostomy pouch and recover its original configuration when released.

### BACKGROUND

The present disclosure pertains to an ostomy pouch. More particularly, the present disclosure pertains to an ostomy pouch with a viewing option window.

Ostomy pouches for collecting body waste are used by patients who have had surgery such as a colostomy, ileostomy, or urostomy. Ostomy pouches typically include flat, opposing side walls secured together along their edges to define a collection cavity. One of the side walls is provided with an opening to receive a stoma, and means to secure the pouch to the user, such as an adhesive barrier, so that body waste discharged through the stoma is received within the cavity.

In order to determine whether an ostomy bag is installed properly or to inspect a stoma, a user or clinician may wish to view the stoma while the ostomy pouch is secured to the user. Similarly, the body waste discharge may also need to be inspected while the ostomy pouch is secured to the user. Thus, it is desirable to provide an ostomy pouch system including an option to view the stoma and interior of the pouch, while still covering the pouch wall for user's discretion.

Accordingly, there is a need for an improved ostomy pouch that allows a view of the stoma or stoma output.

### BRIEF SUMMARY

An ostomy pouch including a viewing option window is provided according to various embodiments.

In one aspect, an ostomy pouch may include a body side wall and a distal wall joined along their peripheral edges to define a cavity therebetween for collecting body waste, an inlet opening defined in the body side wall configured to receive a stoma, and a viewing window member. The viewing window member may be formed from an elastomeric material covering at least a portion of the distal wall. The viewing window member may include a viewing opening having a first configuration. The viewing window member may be configured to be stretched to increase the size of the viewing opening for viewing an interior of the ostomy pouch and recover the first configuration when released.

In an embodiment, at least a portion of the distal wall may be formed from a transparent material, while the viewing window member may be formed from an opaque elastomeric material. For example, the viewing window member may be formed from a thermoplastic elastomer.

In some embodiments, the ostomy pouch may further include a distal comfort panel attached to and covering the distal wall. The distal comfort panel may be formed from an opaque material and include a first opening. In such embodiments, the viewing window member may be arranged, such that the viewing opening is arranged within the first opening.

In an embodiment, the viewing opening may be a circular opening having a diameter smaller than a diameter of the first opening, wherein the viewing window member may be configured to be stretch in various directions to increase the size of the viewing opening and return to the first configuration when released.

In another embodiment, the viewing opening may include a slit-like opening extending across the viewing window member, wherein the viewing window member is configured to be stretched and separated at the slit-like opening to provide a viewing window and return to the first configuration when released. The slit-like opening may be formed from a cut extending across at least a portion of the viewing window member.

In an embodiment, the viewing window member may be arranged between the distal wall and the distal comfort panel, and sealed to the distal comfort panel. In another embodiment, the viewing window member is arranged between the distal wall and the distal comfort panel, and sealed to the distal wall.

In some embodiments, the ostomy pouch may include a curtain formed from an opaque material and configured to cover the viewing opening.

In an embodiment, the curtain may include a first portion attached to the viewing window member and a second unattached portion, wherein the curtain may be configured to be opened by lifting up the second unattached portion to provide an access to the viewing opening.

In another embodiment, the curtain may be integrally formed with the distal comfort panel. For example, the curtain may be formed from a punched-out section of the distal comfort panel, wherein a first portion of the punched-out section remains connected to the comfort panel while a second portion of the punched-out section is unattached from the distal comfort panel. In such an embodiment, the curtain may be configured to be opened by lifting up the second portion.

In an embodiment, the ostomy pouch may include a curtain securing member configured to hold the curtain in a closed position.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is an exploded-view drawing of an ostomy pouch according to an embodiment.

FIG. 2 is a front view of the ostomy pouch of FIG. 1 with a curtain covering an opening of a viewing window member.

FIG. 3 is a front view of the ostomy pouch of FIG. 2 without the curtain.

FIG. 4 is a front view of an ostomy pouch according to another embodiment.

FIG. 5 is a front view of an ostomy pouch according to another embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

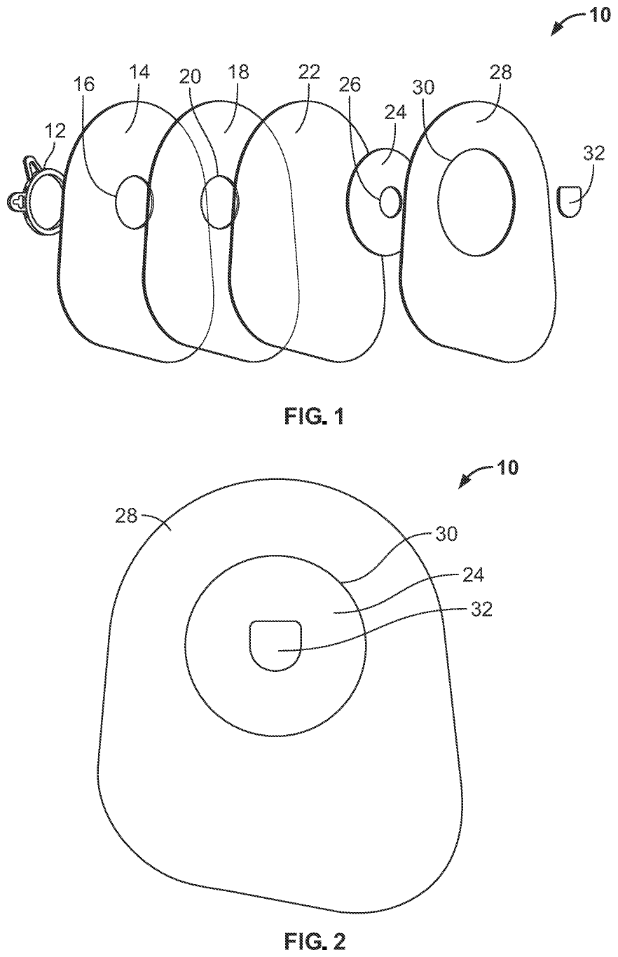

Referring now to the figures, FIGS. 1-3 show an ostomy pouch 10 including a viewing option according to an embodiment. The ostomy pouch 10 may generally include a patient attachment device 12, a body side comfort panel 14, a body side wall 18, a distal wall 22, a viewing window member 24, a distal comfort panel 28, and a curtain 32.

FIG. 1 shows an exploded view of the ostomy pouch 10. The body side wall 18 and the distal wall 22 may be sealed along their peripheral edges to define a cavity therein for collecting stoma output. The body side comfort panel 14 including a first opening 16 may be attached to the body side wall 18. The body side wall 18 may include a second opening 20, wherein the first and second openings 16, 20 may be aligned and configured to receive a stoma. The distal comfort panel 28 may be attached to the distal wall 22.

In this embodiment, the ostomy pouch 10 may be configured as a pouch for a 2-piece ostomy pouch system and the patient attachment device 12 may be a flange that includes a pouch side coupling member. The pouch side coupling member may be configured to engage with a barrier side coupling member (not shown) for securing the ostomy pouch 10 to a skin barrier (not shown). In use, the skin barrier may be attached to a user around a stoma and the ostomy pouch 10 may be attached to the skin barrier by engaging the coupling members, such that the patient attachment device 12 may surround the stoma and stoma output may be received through the first and second openings 16, 18. In other embodiments, the ostomy pouch 10 may be configured as a one-piece ostomy pouch system including a skin barrier attached to a body side of the pouch and the patient attachment device 12 may be a 1-piece pouch with an attached barrier.

The distal wall 22 may be formed from a transparent film to allow viewing of the stoma and body waste collected in the pouch. The distal wall 22 may be covered with the distal comfort panel 28 formed from a suitable opaque material, such as a nonwoven or fabric. The distal comfort panel 28 may include a third opening 30 for viewing.

In this embodiment, the ostomy pouch 10 may include the viewing window member 24, which may be arranged between the distal wall 22 and the distal comfort panel 28. The viewing window member 24 may be configured to cover at least a portion of the distal wall 22 exposed through the third opening 30 to provide discretion for a user. The viewing window member 24 may include a viewing opening 26. In the embodiment of FIG. 1, each of the third opening 30 and the viewing opening 26 may have a generally circular shape, wherein the viewing opening 26 may have a diameter smaller than a diameter of the third opening 30. In other embodiments, the third opening 30 and the viewing opening 26 may be configured to have various shapes, such as rectangular openings, oval openings, and the like, wherein the size of the third opening 30 is greater than the size of the viewing opening 26, such that the viewing opening 26 may be arranged within the third opening 30.

The viewing window member 24 may be configured to be stretched by a user to enlarge the viewing opening 26 for viewing and substantially recover its original configuration when released. In an embodiment, the viewing window member 24 may be formed from a suitable resilient material, such as an elastomeric material. For example, the viewing window member 24 may be formed from a thermoplastic elastomer. In such an embodiment, the viewing window member 24 may be configured such that the viewing opening 26 may be stretched in various directions for desired viewing of the stoma or stoma output and spring back to the original size and configuration of the viewing opening 26 when released. The viewing window member 24 may be heat sealed to the distal wall 22 and/or the distal comfort panel 28. In another embodiment, the viewing window member 24 may be attached to a distal side of the distal comfort panel 28.

In the embodiment of FIGS. 1-3, the ostomy pouch may include the curtain 32 configured to cover the viewing opening 26. The curtain 32 may be separately formed from a suitable opaque material and attached to the viewing window member 24 to cover the viewing opening 26, such that a user may open the curtain 32 to access the viewing opening 26. In an embodiment, a portion of the curtain 32 may be sealed to the viewing window member 24 above the viewing opening 26 and configured to be opened by lifting up an unattached portion of the viewing window member 24.

FIG. 2 shows a front view of the ostomy pouch 10, wherein the curtain 32 is in a closed position covering the viewing opening 26 to prevent viewing of the stoma and pouch content through the viewing opening 26. In the closed position, the distal surface of the distal wall 22 may be completed covered by the distal comfort panel 28, the viewing window member 24, and the curtain 32.

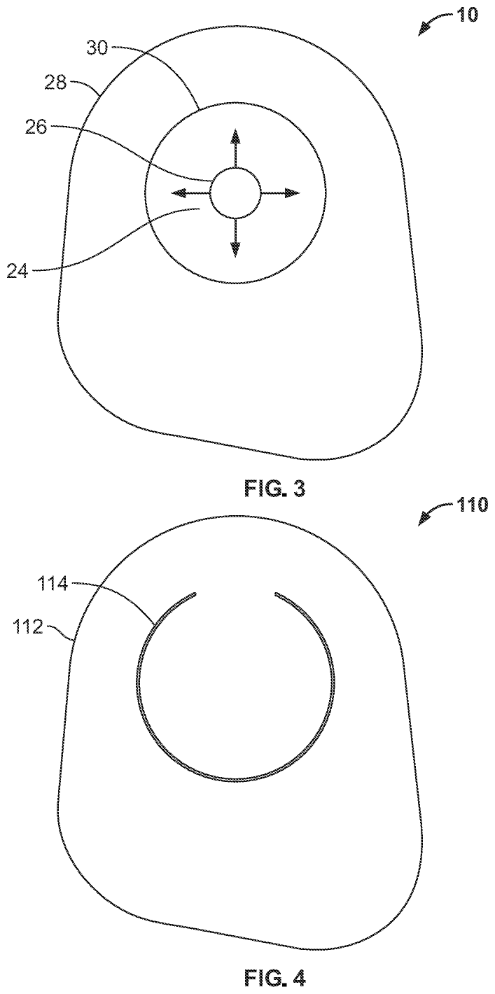

FIG. 3 shows a front view of ostomy pouch 10 without the curtain 32. The viewing window member 24 may be configured to allow a user to manually manipulated the viewing window member 24 to increase the size of the viewing opening 26 for viewing. For example, viewing window member 24 may be stretched in various directions, as shown by arrows on FIG. 3. The viewing window member 24 may be configured to constrict back to its original configuration when released.

FIG. 4 shows a front view of an ostomy pouch 110 according to another embodiment. The ostomy pouch 110 may be configured similar to ostomy pouch 10 of FIG. 1 generally including a body side wall 18, a distal wall 22, a viewing window member 24, and a distal comfort panel 112, except in this embodiment, a curtain 114 may be integrally formed with the distal comfort panel 112. In this embodiment, the curtain 114 may be formed from a punched-out section of the distal comfort panel 112, wherein a portion of the curtain 114 remains attached to the distal comfort panel 112 while the other portion is unattached for opening of the curtain 114. The viewing window member 24 may be arranged under the curtain 114 and exposed when the curtain 114 is opened. For example, the curtain 114 may be lifted up, moved away from the distal comfort panel 112, such that a user may access the viewing window member 24 to stretch and enlarge the viewing opening 26 to view the stoma or stoma output.

In an embodiment, the ostomy pouch 110 may include a curtain securing member (not shown) configured to maintain the curtain 114 in a closed position to prevent the curtain 114 from opening accidentally. For example, the curtain securing member may include a first fastener provided on the viewing window member 24 and a second fastener provided on the curtain 114, wherein the first and second fasteners may be configured to engage with each other to hold the curtain in a closed position.

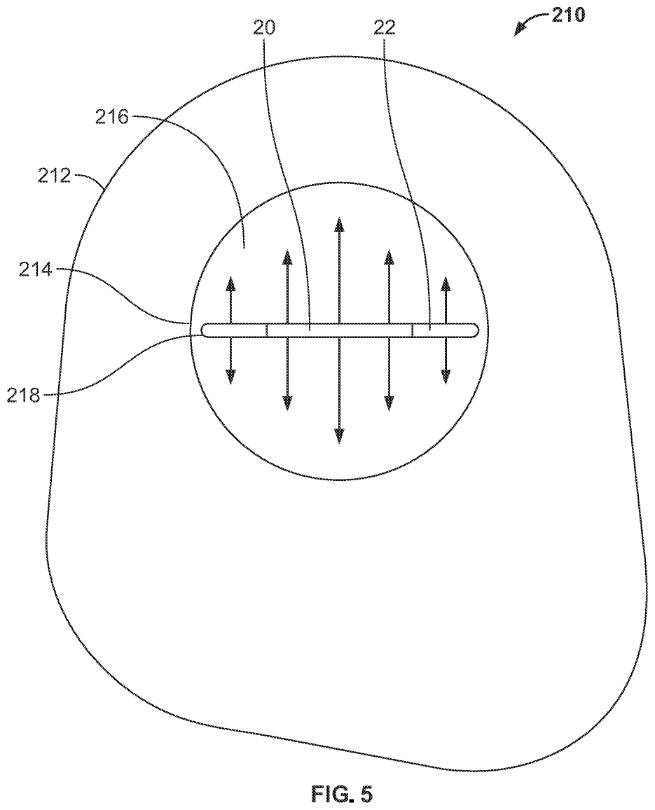

FIG. 5 shows a front view of an ostomy pouch 210 according to an embodiment. The ostomy pouch 210 may be configured similar to ostomy pouch 10 of FIG. 1 generally including a body side wall 18, a second opening 20, a distal wall 22, a viewing window member 216, and a distal comfort panel 212, except in this embodiment, the viewing opening 26 may be defined by a slit-like opening 218. The distal comfort panel 212 may include a third opening 214 for viewing.

In this embodiment, the slit-like opening 218 may be formed as a relatively thin opening that extends horizontally across the viewing window member 216. In another embodiment, the slit-like opening 218 may be formed as a horizontally extending cut in the viewing window member 216. In other embodiments, the slit-like opening 218 may be formed as a relatively thin opening that extends vertically across the viewing window member 216 or a vertically extending cut in the viewing window member 216. As it was with the viewing window member 24, the viewing window member 216 may be configured to be a resilient member which may be manipulated by a user to increase the size of the viewing opening 218 and recover its original shape and size when released. In an embodiment, the viewing window member 216 may be stretched in a direction perpendicular to the slit-like opening 218 to separate the viewing window member 216 at the slit-like opening 218 for viewing. For example, the slit-like opening 218 may be stretched to open in the directions shown by arrows in FIG. 5.

In an embodiment, ostomy pouch 210 may include a curtain (not shown) configured to cover the slit-like opening 218. The curtain may be attached to the distal comfort panel 212. For example, the curtain may be configured sufficiently wide to cover the entire slit-like opening 218. From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

1. An ostomy pouch comprising:
a body side wall and a distal wall joined along their peripheral edges to define a cavity therebetween for collecting body waste;
an inlet opening defined in the body side wall configured to receive a stoma; and
a viewing window member formed from an elastomeric material covering at least a portion of the distal wall, wherein the viewing window member includes a viewing opening having a first configuration, and wherein the viewing window member is configured to be stretched to increase a size of the viewing opening for viewing an interior of the ostomy pouch and recover the first configuration when released.

2. The ostomy pouch of claim 1, wherein at least a portion of the distal wall is formed from a transparent material and the viewing window member is formed from an opaque elastomeric material.

3. The ostomy pouch of claim 1, wherein the viewing window member is formed from a thermoplastic elastomer.

4. The ostomy pouch of claim 1, wherein the ostomy pouch further includes a distal comfort panel attached to and covering the distal wall, wherein the distal comfort panel is formed from an opaque material and includes a first opening, and wherein the viewing window member is arranged such that the viewing opening is arranged within the first opening.

5. The ostomy pouch of claim 4, wherein the viewing opening is a circular opening having a diameter smaller than a diameter of the first opening, wherein the viewing window member is configured to be stretch in various directions to increase the size of the viewing opening and return to the first configuration when released.

6. The ostomy pouch of claim 4, wherein the viewing opening comprises a slit-like opening extending across the viewing window member, wherein the viewing window member is configured to be stretched and separated at the slit-like opening to provide a viewing window and return to the first configuration when released.

7. The ostomy pouch of claim 6, wherein the slit-like opening is formed from a cut extending across at least a portion of the viewing window member.

8. The ostomy pouch of claim 4, wherein the viewing window member is arranged between the distal wall and the distal comfort panel, wherein the viewing window member is sealed to the distal comfort panel.

9. The ostomy pouch of claim 4, wherein the viewing window member is arranged between the distal wall and the distal comfort panel, wherein the viewing window member is sealed to the distal wall.

10. The ostomy pouch of claim 1, wherein the ostomy pouch includes a curtain formed from an opaque material and configured to cover the viewing opening.

11. The ostomy pouch of claim 10, wherein the curtain includes a first portion attached to the viewing window member and a second unattached portion, wherein the curtain is configured to be opened by lifting up the second unattached portion to provide an access to the viewing opening.

12. The ostomy pouch of claim 10, wherein the curtain is integrally formed with the distal comfort panel.

13. The ostomy pouch of claim 12, wherein the curtain is formed from a punched-out section of the distal comfort panel, wherein a first portion of the punched-out section remains connected to the comfort panel while a second portion of the punched-out section is unattached from the distal comfort panel, and wherein the curtain is configured to be opened by moving the second portion.

14. The ostomy pouch of claim 10, wherein the ostomy pouch includes a curtain securing member configured to hold the curtain in a closed position.
